(19) United States
(12) Patent Application Publication
(10) Pub. No.: US 2008/0134487 A1
(43) Pub. Date: Jun. 12, 2008

(54) Integral waistband for a disposable absorbent article, an article and a method of forming said article

(76) Inventor: Julia Hartono, Menasha, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/635,883

(22) Filed: Dec. 7, 2006

(51) Int. Cl. A61F 13/49; B23P 11/00

(52) U.S. Cl. 29/428; 604/396; 604/385.3

(57) ABSTRACT

An integral waistband for a disposable absorbent article having a waist opening is disclosed, along with a disposable absorbent article and a method of forming the article. The integral waistband includes an inner layer, an outer layer, and at least two elastic members positioned therebetween. Each of the inner and outer layers has a first end and a first surface. The inner and outer layers are bonded together such that the first end of the inner layer initially extends beyond the first end of the outer layer and the portion of the first surface of the inner layer bonded to the first surface of the outer layer forms a laminate. The remainder of the inner layer is a non-laminate. The non-laminate is folded over the outer layer and is bonded thereto to form an integral waistband located on the outer layer away from the waist opening.

### BACKGROUND OF THE INVENTION

A disposable absorbent article is designed for absorbing human exudate. The disposable absorbent article is similar in appearance, size and shape to a regular cloth underwear except that it is not designed to be laundered and reused two or more times. A disposable absorbent article is intended to be worn by persons, including infants, toddlers, or adults, and is designed for single or temporary use. The disposable absorbent article is meant to be disposed of after being used once. The disposable absorbent article is designed to be pulled up around the user's torso without having to first open the garment in order to place it on a person's body. The stretchability of the material used to construct the disposable absorbent article permits the garment to snugly conform to the anatomy of the user's torso. The disposable absorbent article can be manufactured to be an infant diaper, a child training pant, an adult incontinence garment, a feminine menstrual pant, etc.

Some disposable absorbent articles manufactured today resemble regular cloth underwear in that they have a waist opening and a pair of leg openings. Such disposable absorbent articles can be pulled up around the torso of a user in a similar fashion as regular cloth underwear. It has been found that a disposable absorbent article is generally more comfortable to wear and more aesthetically pleasing to the user when the disposable absorbent article contains an integral waistband. One reason for this is that regular cloth underwear, which adult incontinent users have been wearing for decades, contains a waistband. Therefore, adult incontinent users have indicated that they would like to see a waistband on their disposable absorbent articles. An integral waistband is cost effective for a manufacturer to produce and provides an aesthetic appearance similar to waistbands formed on regular cloth underwear. The integral waistband also functions to provide sufficient tension to firmly hold the disposable absorbent article about the user's torso even when the article is insulted with a large amount of body fluid and/or excrement.

Now an integral waistband for a disposable absorbent article has been invented, along with a disposable absorbent article and a method of forming the article.

### SUMMARY OF THE INVENTION

Briefly, this invention relates to an integral waistband for a disposable absorbent article having a waist opening. The invention also relates to a disposable absorbent article having an integral waistband and a method of forming the article. The integral waistband includes an inner layer, an outer layer, and at least two elastic members positioned therebetween. Each of the inner and outer layers has a first end, a second end and a first surface. The inner and outer layers are bonded together such that the first end of the inner layer initially extends beyond the first end of the outer layer. The portion of the first surface of the inner layer bonded to the first surface of the outer layer forms a laminate and the remainder of the inner layer is a non-laminate. The non-laminate is folded over the first end of the outer layer and is bonded thereto to form an integral waistband having a visible edge located away from the waist opening.

The disposable absorbent article having an integral waistband includes a front region, a back region and an absorbent assembly secured to the front and back regions. The front and back regions are joined together by a pair of seams to form a unitary article having a waist opening. The front region includes an inner layer, an outer layer, and at least two elastic members positioned therebetween. Each of the inner and outer layers has a first end, a second end and a first surface. The inner and outer layers are bonded together such that the first end of the inner layer initially extends beyond the first end of the outer layer. The portion of the first surface of the inner layer bonded to the first surface of the outer layer forms a laminate and the remainder of the inner layer is a non-laminate. The non-laminate is folded over the first end of the outer layer and is bonded thereto to form an integral waistband having a visible edge located on the outer layer and away from the waist opening.

The method includes the steps of forming the front and back regions, securing an absorbent assembly thereto and joining the front and back regions together by a pair of seams to form a unitary article having a waist opening and a pair of leg openings. The non-laminate is then folded over a portion of the outer layer of the back region to form an integral waistband having a visible edge located on the outer layer and away from said waist opening.

### BRIEF DESCRIPTION OF THE DRAWINGS

FIG. 1 is a perspective view of a disposable absorbent article having an integral waistband formed thereon.

FIG. 2 is a plane view of a disposable absorbent article before the pair of seams are formed showing a front region, a back region and an absorbent assembly secured thereto and showing an integral waistband formed on the outer layers of both the front and back regions which does not extend into the pair of seams.

FIG. 3 is a side view of the disposable absorbent article shown in FIG. 2.

FIG. 4 is a cross-sectional view of an integral waistband formed from an inner layer, an outer layer and at least two elastic members positioned therebetween, before the inner layer is folded over the first end of the outer layer.

FIG. 5 is a cross-sectional view of the integral waistband shown in FIG. 4 after a portion of the inner layer is folded and bonded to the outer layer.

FIG. 6 is a partial view of a disposable absorbent article showing an alternative embodiment wherein the elastic members of the integral waistband extend into the pair of seams.

FIG. 7 is a cross-sectional view of the integral waistband shown in FIG. 5 except that the five elastic members located adjacent to the fold are of a different color than the remaining elastic members.

FIG. 8 is a cross-sectional view of another embodiment of an integral waistband formed from an inner layer, an outer layer and a plurality of elastic members positioned therebetween and before the inner layer is folded over the first end of the outer layer, and wherein the elastic members are spaced apart in two groups.

FIG. 9 is a cross-sectional view of the integral waistband shown in FIG. 8 after a portion of the inner layer is folded and bonded to the outer layer.

FIG. 10 is a cross-sectional view of still another embodiment of an integral waistband formed from an inner layer, an outer layer and a plurality of elastic members positioned therebetween and before the inner layer is folded over the first end of the outer layer, and wherein two elastic members are secured to the extended portion of the inner layer.

FIG. 11 is a cross-sectional view of the integral waistband shown in FIG. 10 after a portion of the inner layer is folded and bonded to the outer layer.

### DETAILED DESCRIPTION

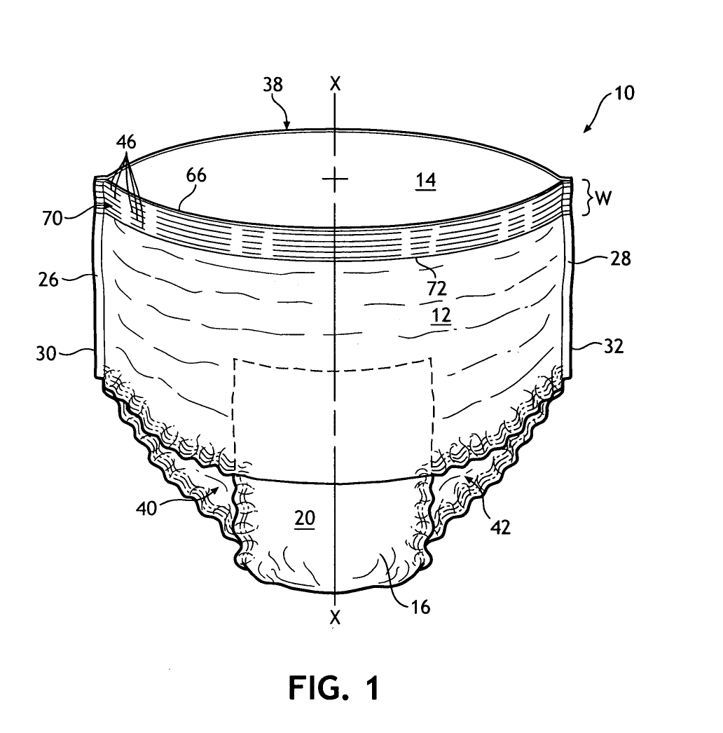

Referring to FIG. 1, a disposable absorbent article 10 is depicted. The disposable absorbent article 10 is intended to be worn by persons, including infants, toddlers, or adults, and is designed for a single or temporary use. The disposable absorbent article 10 is meant to be disposed of after being used once instead of being laundered or dry cleaned for re-use. The disposable absorbent article 10 can be a unitary undergarment which is designed to be pulled up around the user's torso without having to first open the disposable absorbent article 10 in order to place it on a person's body. In FIG. 1, the disposable absorbent article 10 is shown as an adult incontinent undergarment solely for discussion purposes. The disposable absorbent article 10 appears in FIG. 1 as it looks just prior to being pulled up around a user's torso.

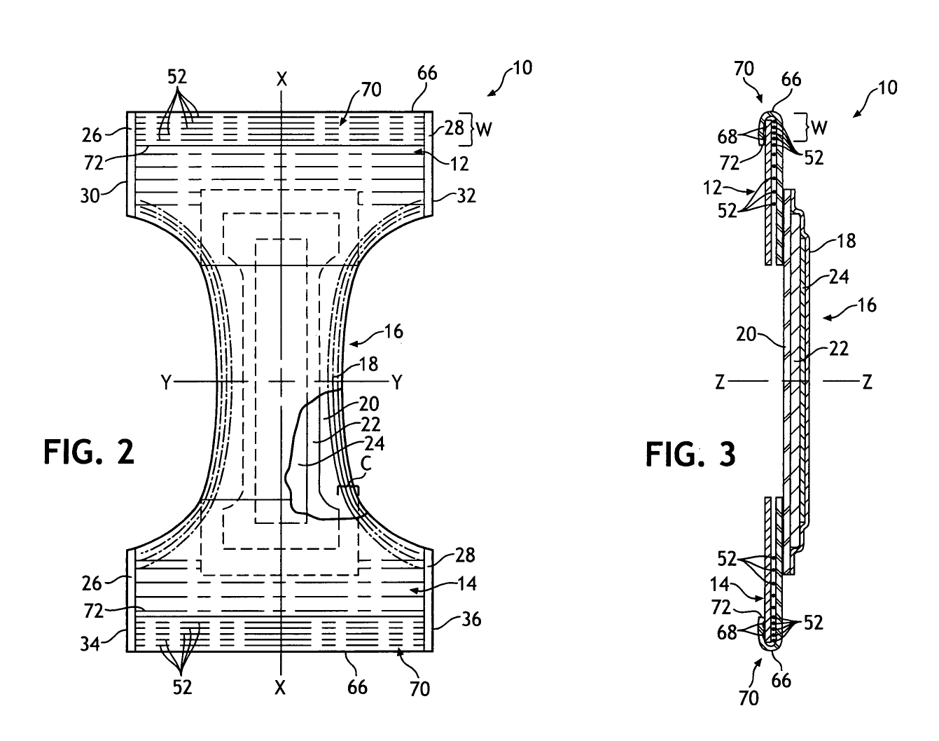

Referring to FIGS. 2 and 3, the disposable absorbent article 10 is depicted in an open configuration solely for the purpose of better showing the various components. In FIG. 2, the open, disposable absorbent article 10 is shown having a longitudinal central axis X-X and a transverse central axis Y-Y. In FIG. 3, the open, disposable absorbent article 10 is shown having a vertical central axis Z-Z. The disposable absorbent article 10 includes a front region 12, a back region 14 and an absorbent assembly 16 secured to the front and back regions, 12 and 14 respectively. The absorbent assembly 16 bridges the front and back panels, 12 and 14 respectively, to form a three-piece chassis. It should be known to those skilled in the art that the disposable absorbent article 10 could be constructed with a single chassis or be made of two or more chassis pieces. In FIG. 1, the front region 12 is considered a chassis piece, the back region 14 is considered to be a chassis piece and the absorbent assembly 16 is considered to be a chassis piece.

Still referring to FIGS. 2 and 3, the absorbent assembly 16 can be secured to the front and back regions, 12 and 14 respectively, by various attachment mechanisms. Examples of some of the attachment mechanisms that can be used include but are not limited to: an adhesive, heat, pressure, heat and pressure, an ultrasonic bond, a chemical bond, various types of mechanical fasteners, such as sewing or stitching, or by other means known to those skilled in the art. Desirably, the absorbent assembly 16 is secured to the front and back regions, 12 and 14 respectively, by a hot melt adhesive. The absorbent assembly 16 is shown having a liquid permeable bodyside liner 18, a liquid-impermeable backsheet 20 and an absorbent 22 positioned therebetween. The bodyside liner 18 can be formed from a material that will allow liquids, especially urine, to pass quickly therethrough. The bodyside liner 18 can be formed from natural or synthetic materials that are either woven or non-woven. Spunbond is a nonwoven material that functions well as the bodyside liner 18. Spunbond is manufactured and sold by Kimberly-Clark Corporation, having an office at 401 North Lake Street, Neenah, Wis. 54956.

The backsheet 20 can be formed from any material that will limit or prevent the passage of a liquid therethrough. Desirably, the backsheet 20 is liquid-impermeable and will prevent liquids, especially urine, from passing therethrough. Some materials that function well as a backsheet 20 include synthetic materials, such as plastic and thermoplastic materials, especially films; polyolefins, such as polyethylene and polypropylene; and other materials known to those skilled in the art. The backsheet 20 can be relatively thin, having a thickness of only a few millimeters, and should have a soft hand or feel and be flexible. A film material that is quiet when subjected to external forces works best. More desirably, the backsheet 20 is liquid-impermeable but vapor permeable. By being “vapor permeable” the backsheet 20 will be able to allow air, vapor and gases to exit from the absorbent 22. By increasing the breathability of the backsheet 20, one can design a more effective disposable absorbent article 10.

The absorbent 22 can be formed from natural or synthetic materials. The absorbent 22 can be made from cellulosic fibers, wood pulp, textile fibers or from other absorbent materials known to those skilled in the art. Superabsorbents, commonly in solid form and in the shape of small particles, granules, flakes, etc., can be mixed, combined, attached, printed or otherwise added to the absorbent material to increase the absorbent capacity of the absorbent 22. A surge layer 24 can also be optionally used. The surge layer 24 is normally positioned between the bodyside liner 18 and the absorbent 22. The surge layer 24 functions to rapidly acquire and temporarily retain body fluid, such as urine, before it can be absorbed into the absorbent 22. Desirably, the surge layer 24 is capable of wicking body fluid lengthwise and/or widthwise across its surface, as well as directing the body fluid downward in a z-direction, into the absorbent 22. The surge layer 24 will limit rewet from occurring. Rewet is the ability of body fluid that has insulted the disposable absorbent article 10 to flow back out when the disposable absorbent article 10 is squeezed or contorted by body movement. A low rewet value is most advantageous for it assures that little or no body fluid will be able to be forced back out of the disposable absorbent article 10 and against the skin of the user.

It should be noted that the absorbent assembly 16 can be constructed with two or more layers of absorbent 22, if desired. Any or all of the layers of absorbent 22 can contain a superabsorbent material. Likewise, the absorbent assembly 16 can also be constructed without the bodyside liner 18 or without the backsheet 20, if one so desires.

Referring again to FIGS. 1 and 2, the disposable absorbent article 10 further includes a pair of seams 26 and 28 that secure the front region 12 to the back region 14. The pair of seams 26 and 28 can be formed by various means known to those skilled in the art. Most desirably, the pair of seams 26 and 28 is formed by an ultrasonic bond. The pair of seams 26 and 28 join first and second side edges, 30 and 32 respectively, of the front region 12 to first and second side edges, 34 and 36 respectively, of the back region 14. Once the pair of seams 26 and 28 is formed, the disposable absorbent article 10 acquires a unitary design having a waist opening 38 and a pair of leg openings 40 and 42. By a “unitary design” it is meant that the disposable absorbent article 10 functions similarly to regular cloth underwear in that it has to be pulled up along the wearer's legs and around the wearer's torso. The pair of seams 26 and 28 is not designed to be opened and closed. Instead, the pair of seams 26 and 28 can be opened once by breaking or destroying the bond that forms each seam. Once the pair of seams 26 and 28 is broken, the disposable absorbent article 10 must be discarded and replaced since the disposable absorbent article 10 does not contain a refastening mechanism.

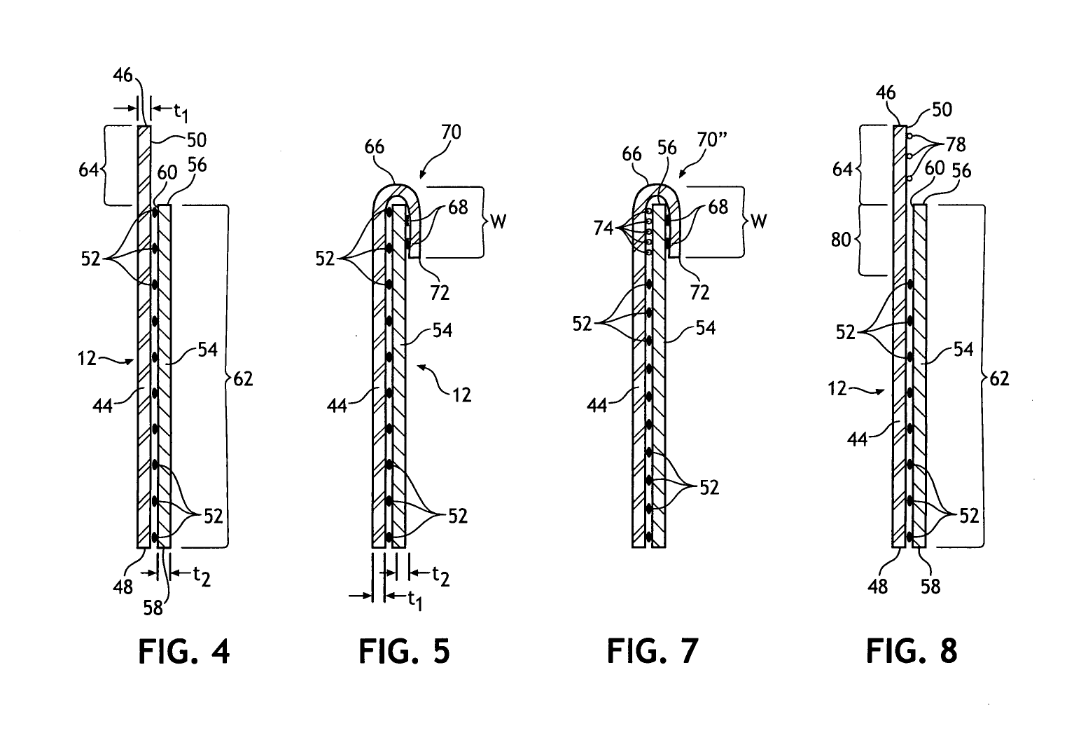

Turning now to FIGS. 4 and 5, the front and/or back regions, 12 and/or 14 respectively, can be formed from an inner layer 44 having a first end 46, a second end 48 and a first surface 50. The first surface 50 of the inner layer 44 faces away from the skin of the wearer of the disposable absorbent article 10. The inner layer 44 should have a soft hand or feel since it will be in contact with the wearer's body. The inner layer 44 should also be stretchable to allow it to conform to the wearer's torso during various body movements. The inner layer 44 should also be breathable. By “breathable” it is meant that the inner layer 44 will allow air, vapors and gases to easily pass therethrough. A good material from which the inner layer 44 can be constructed is spunbond. Other materials that could also be used include woven and non-woven webs of natural materials (e.g., wood or cotton fibers), woven and non-woven webs of synthetic materials, and webs formed from a combination of natural and synthetic materials. The inner layer 44 can be treated or coated to make it more hydrophilic, if desired. Desirably, the inner layer 44 is a liquid permeable, breathable and stretchable non-woven material.

The inner layer 44 should also be relatively thin, having a thickness t₁ of less than about 5 mm. Desirably, the thickness t₁ of the inner layer 44 is less than about 4 mm, and more desirably, the thickness t₁ is less than about 3 mm. When the inner layer 44 is formed from spunbond having a thickness t₁ of less than about 5 mm, the material will permit a color, especially a darker color, to show through. Additional information on this point will be explained shortly.

At least two elastic members 52 are positioned adjacent to at least a portion of the first surface 50 of the inner layer 44. The number of elastic members 52 can range from between 2 to about 200. A plurality of elastic members 52 can be employed. By “plurality” it is meant at least three elastic members 52. Desirably, from between 3 to 50 elastic members are present. More desirably, at least four elastic members 52 are present. The elastic members 52 can be in the form of elastic strands spaced apart from each other. For example, each of the elastic members 52 can be spaced from between about 0.5 to about 25 millimeters (mm) apart. Desirably, each of the elastic members 52 can be spaced from between about 1 to about 10 millimeters (mm) apart. The exact spacing or distance between adjacent elastic members 52 can vary depending upon their location within the front and/or back regions, 12 and 14 respectively. All of the elastic members 52 do not have to be equally spaced apart from an adjacent elastic member 52. For example, the elastic members 52 located closest to the waist opening 38 and/or to the leg openings 40 and 42 can be spaced closer together than the elastic members 52 situated therebetween.

The individual elastic members 52 can be formed from CREORA SPANDEX, LYCRA, FULFLEX or from any other elastomeric material known to those skilled in the art. CREORA SPANDEX is a registered trademark of Hyosung Corporation, having an office at 450, Gongdeok-dong, Mapo-gu, Seoul, Korea (121-720). LYCRA is a registered trademark of Invista, having an office at 4123 East 37^{th} St. North, Wichita, Kans. 67220. FULFLEX is a registered trademark of Fulflex Elastomerics Worldwide, having an office at 36 Beach Street, Westerly, R.I. 02891. The individual elastic members 52 should be aligned approximately parallel to one another or be aligned at a slight angle or be skewed relative to one another. However, the elastic members 52 should not overlap, cross or intersect one another. Desirably, the elastic members 52 will extend transversely across the front and/or back regions, 12 and/or 14 respectively. The elastic members 52 can extend into the pair of seams 26 and 28, or alternatively, they can stop short of entering the pair of seams 26 and 28.

The elastic members 52 can vary in shape, size, configuration and/or length. The diameter and/or cross-sectional configuration of the elastic members 52, the decitex (weight in grams per 10,000 meters of an elastic member) of the elastic members 52, and the tension imparted into each of the elastic members 52 can all be varied to suit one's particular needs. Tension can be controlled by varying a number of items such as the spacing between each elastic member 52, the material from which the elastic members 52 are formed, the number of elastic members 52 that are present, the stretch imparted into each of the elastic members 52 before they are secured to the front and back regions, 12 and 14 respectively, the decitex of each of the elastic members 52, the cross-sectional area of each of the elastic members 52, the mechanism used to secure each of the elastic members 52 to the inner layer 44, etc. One skilled in the art will know how these and other variables affect the tension value of the elastic members 52.

All of the elastic members 52 do not have to have the same size, decitex, tension, etc. Each elastic member 52 can have any desired cross-sectional profile. Desirably, each elastic member 52 has a round or circular cross-sectional configuration but almost any geometrical cross-sectional configuration could be used. Some other geometrical cross-sectional configurations include: square, rectangular and oval. The elastic members 52 can also be in the form of elastic ribbons having a square or rectangular cross-sectional configuration. Furthermore, the elastic members 52 can be in the form of fabric ribbons having a square or rectangular cross-sectional configuration of various dimensions.

The various ways of positioning, orienting and/or adhering the individual elastic members 52 in between the inner and outer layers, 44 and 54 respectively, are well known to those skilled in the art. For example, each of the elastic members 52 can be at least partially coated with an adhesive and then be secured to at least a portion of the first surface 50 of the inner layer 44. Alternatively, at least a portion of the first surface 50 of the inner layer 44 can be at least partially coated with an adhesive, such as by slot coating or by a spiral spray, and then the elastic members 52 can be secured to the adhesive. Other ways of securing the elastic members 52 to the inner and outer layers, 44 and 54 respectively, are well known to those skilled in the art.

For the elastic members 52 located adjacent to the waist opening 38, the spacing or distance between two adjacent elastic members 52 can range from between about 1 mm to about 12.5 mm. Desirably, the spacing or distance between two adjacent elastic members 52 can range from between about 2 mm to about 10 mm. More desirably, the spacing or distance between two adjacent elastic members 52 can range from between about 3 mm to about 8 mm. Even more desirably, the spacing or distance between two adjacent elastic members 52 can be less than about 5 mm.

Returning to FIGS. 1-3, the number of elastic members 52 that are present in each of the front and/or back regions, 12 and/or 14 respectively, can vary. Typically, the front and/or back regions, 12 and 14 respectively, will contain from 1 to about 10 waist elastic members which are aligned relatively close to the waist opening 38. The front and/or back regions, 12 and 14 respectively, will also contain from 1 to about 6 leg elastic members that are aligned relatively close to each of the leg openings 40 and 42. In addition, the front and/or back regions, 12 and 14 respectively, can contain from 1 to about 200 chassis elastic members that are located between the waist elastic members and the leg elastic members.

Returning again to FIGS. 4 and 5, the front and/or back regions, 12 and 14 respectively, of the disposable absorbent article 10 further include an outer layer 54 having a first end 56, a second end 58 and a first surface 60. The outer layer 54 is spaced away from the body of the wearer of the disposable absorbent article 10. The outer layer 54 can be of the same material as the inner layer 44 or it can be made from a different material. The outer layer 54 can have a soft hand or feel and should be formed from a material that is quiet. A quiet material is one that will not emit discernible, audible sounds as the material is subjected to stresses and strains caused by body movements of the wearer of the disposable absorbent article 10. The outer layer 54 should also be stretchable to allow it to conform to the wearer's torso during various body movements. The outer layer 54 should be breathable to allow air, vapors and gases to easily pass therethrough. A good material from which the outer layer 54 can be constructed is spunbond. Other materials that could also be used include those listed above for the inner layer 44. Desirably, the outer layer 54 is a liquid permeable, breathable and stretchable non-woven material.

The outer layer 54, like the inner layer 44, should be relatively thin, having a thickness t₂ of less than about 5 mm. Desirably, the thickness t₂ of the outer layer 54 is less than about 4 mm, and more desirably, the thickness t₂ is less than about 3 mm. When the outer layer 54 is formed from spunbond having a thickness t₂ Of less than about 5 mm, the material will permit a color, especially a darker color, to show therethrough. Additional information on this point will be explained shortly.

The entire first surface 60 of the outer layer 54 is secured, adhered or bonded to a portion of the inner layer 44. The inner and outer layers, 44 and 54 respectively, can be secured together by an adhesive, by ultrasonics, by heat, by pressure, by heat and pressure, or by other means known to those skilled in the art. However, a portion of the inner layer 44 is not initially secured, adhered or bonded to the outer layer 54. Instead, this non-bonded portion will extend beyond the first end 56 of the outer layer 54. In FIG. 4, the first end 46 of the inner layer 44 initially extends beyond the first end 56 of the outer layer 54. Desirably, the inner layer 44 is at least about 12 mm longer than the outer layer 54. When the inner and outer layers, 44 and 54 respectively, are bonded together, the area where the two layers 44 and 54 overlap form a laminate 62. The remainder of the inner layer 44, denoted as 64, is a non-laminate. The non-laminate 64 can have a length that ranges from between about 2 mm to about 100 mm. Desirably, the non-laminate 64 has a length that ranges from between about 3 mm to about 70 mm. More desirably, the non-laminate 64 has a length that ranges from between about 4 mm to about 50 mm. Even more desirably, the non-laminate 64 has a length that ranges from between about 5 mm to about 25 mm.

The non-laminate 64 is designed to be folded over the first end 56 of the outer layer 54 to form a fold 66. The first end 46 of the inner layer 44 is also the distal end of the non-laminate 64. This first end 46 will overlap a portion of the outer layer 54 and the non-laminate 64 can be secured thereto by an attachment mechanism 68. The attachment mechanism 68 can be an adhesive, an ultrasonic bond, a chemical bond, a heat bond, pressure bond, a heat and pressure bond, an embossment or any combination of the above. As the non-laminate 64 overlaps a portion of the outer layer 54, it will also overlap at least one of the elastic members 52. Desirably, the non-laminate 64 will overlap from between 1 to 10 of the elastic members 52. More desirably, the non-laminate 64 will overlap from between 2 to 6 of the elastic members 52. Even more desirably, the non-laminate 64 will overlap five of the elastic members 52.

Once the non-laminate 64 is folded over and secured to a portion of the outer layer 54, an integral waistband 70 is formed. The integral waistband 70 has a visible edge 72, located on the outer layer 54, which is located away from or distal the fold 66 and the waist opening 38, see FIG. 1. The visible edge 72 will accentuate the presence of the integral waistband 70 and provide it with a relative prominence on the disposable absorbent article 10. The integral waistband 70, in turn, will provide the disposable absorbent article 10 with the appearance and aesthetics of regular cloth underwear. Furthermore, a stitch or design pattern (not shown) can be added to the integral waistband 70 to enhance the visible edge 72 and/or to create a stitch-like or underwear-like waistband seam. The stitch or design pattern can be formed using ultrasonics, heat, pressure, heat and pressure, or by other means known to those skilled in the art.

Referring again to FIGS. 1, 2 and 5, the integral waistband 70 has a width w measured parallel to the longitudinal central axis X-X of the disposable absorbent article 10. The width w of the integral waistband 70 can range from between about 2 mm to about 60 mm. Desirably, the width w of the integral waistband 70 will range from between about 5 to about 50 mm. More desirably, the width w of the integral waistband 70 will range from between about 10 to about 30 mm. Even more desirably, the width w of the integral waistband 70 will range from between about 12 to about 25 mm. The integral waistband 70 can extend across at least a portion of the front region 12, can extend across at least a portion of the back region 14, or can extend across at least a portion of both of the front and back regions, 12 and 14 respectively. Alternatively, the integral waistband 70 can extend completely across the front region 12 between the pair of seams 26 and 28, or it can extend completely across the back region 14 between the pair of seams 26 and 28. As depicted in FIGS. 1 and 2, the integral waistband 70 extends completely across both the front and back regions, 12 and 14 respectively, between the pair of seams 26 and 28. Desirably, the integral waistband 70 will extend transversely across one or both of the front and back regions, 12 and 14 respectively. More desirably, the integral waistband 70 will extend transversely across both of the front and back regions, 12 and 14 respectively.

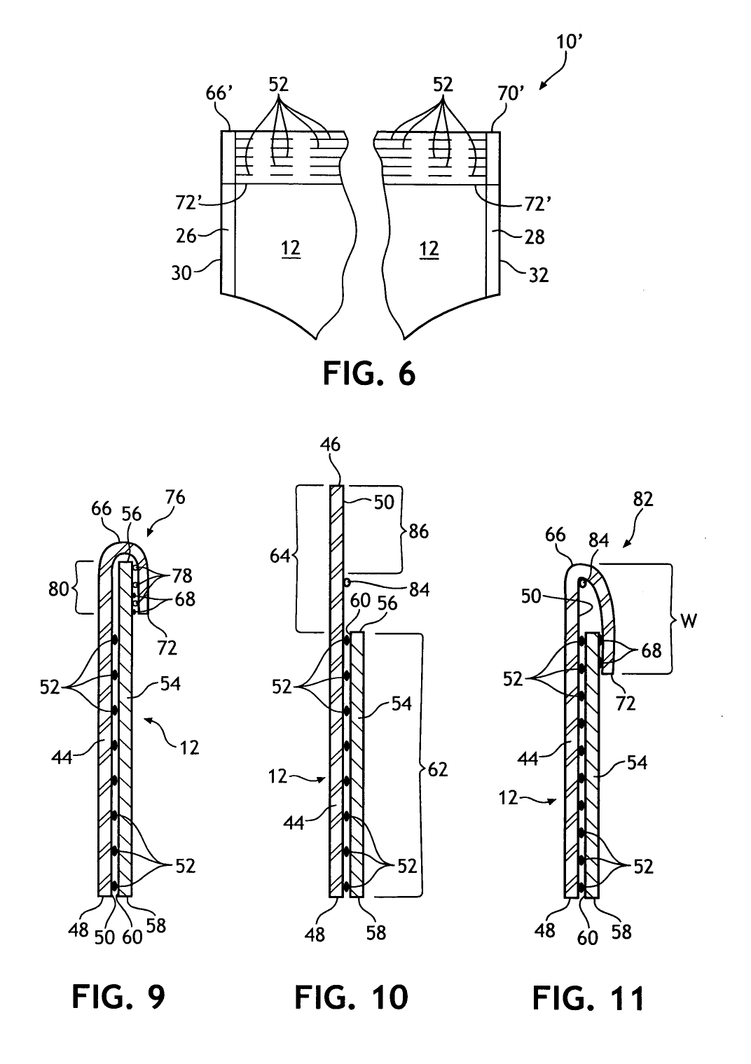

Referring now to FIG. 6, a cross-sectional view of an alternative embodiment of the front region 12 of a disposable absorbent article 10′ is shown. In this embodiment, the elastic members 52 extend transversely across the front region 12 from side edge 30 to the opposite side edge 32. In this embodiment, the elastic members 52 overlap the pair of seams 26 and 28 to form an integral waistband 70′. The integral waistband 70′ has a fold 66′ and a visible edge 72′. It is also possible to apply the elastic members 52 across the back region 14 such that they extend from the side edge 34 to the opposite side edge 36. In so doing, the elastic members 52 will overlap the pair of seams 26 and 28 in the back region 14 to form an integral waistband 70′. When the elastic members 52 are so arranged, the integral waistband 70′ will surround the entire waist opening 38.

Referring now to FIG. 7, the front region 12 of a disposable absorbent article 10 is shown having an integral waistband 70″. The integral waistband 70″ differs from the integral waistband 70 in that elastic members 74 of a different color than the elastic members 52 are located closest to the first end 56 of the outer layer 54. The actual number of elastic members 74 that are of a different color can vary from between 1 to about 10. Desirably, from 1 to 6 different colored elastic members 74 are present. More desirably, from 2 to 5 different colored elastic members 74 are present. In FIG. 7, five different colored elastic members 74 are shown being aligned closest to the first end 56 of the outer layer 54. These five elastic members 74 are of a different color than the remaining elastic members 52. The elastic members 74 can be of a darker or a lighter color than the other elastic members 52. For example, the elastic members 74 can be blue in color while the remaining elastic members 52 are white.

One benefit of using one or more colored elastic members 74 located adjacent to the first end 56 of the outer layer 54 is that this color will be visible through the integral waistband 70″. As mentioned above, the inner and outer layers, 44 and 54 respectively, are relatively thin and one can notice the color of the elastic members 52 and 74 situated therebetween. This is especially true when the inner and outer layers, 44 and 54 respectively, are made from spunbond having a thickness, t₁ and t₂ respectively, of less than about 5 mm. Since the inner and outer layers, 44 and 54 respectively, are relatively thin, the material is somewhat transparent and allows the color of the elastic members 52 and 74 to show through. This is an important feature, for it allows the integral waistband 70, 70′ and 70″ to be more prominent relative to the remainder of the front and back regions, 12 and 14 respectively, of the disposable absorbent article 10 or 10′. For example, most disposable absorbent articles 10 and 10′ are white and colored waistbands would definitely standout and grab the attention of the user.

It should also be noted that the non-laminate 64 of the integral waistband 70, 70′ and 70″ can also be formed from a colored material or be printed, painted, dyed, coated, sprayed, etc. to have a different color than the remainder of the disposable absorbent article 10 or 10′. Ink jet printing is one method known to those skilled in the art that can be used to color the integral waistband 70, 70′ or 70″. Embossing can also be used to distinguish the integral waistband 70, 70′ or 70″ from the remainder of the disposable absorbent article 10 or 10′. An embossing pattern can also be highlighted with one or more colors.

Returning to FIGS. 4 and 5, it should be noted that the laminate 62 forming the front and back regions, 12 and 14 respectively, is breathable. Likewise, the integral waistband 70, 70′ or 70″ is also breathable. However, the laminate 62 is more breathable than the integral waistband 70 since it is formed from one less layer. In essence, the integral waistband 70 has a breathability value which is within about 50% of the breathability value of the laminate 62. Desirably, the integral waistband 70 has a breathability value which is within about 60% of the breathability value of the laminate 62. More desirably, the integral waistband 70 has a breathability value which is within about 65% of the breathability value of the laminate 62. Breathability can be determined using ASTM test 3806, which measures water vapor transmission rate. The water vapor transmission rate test measures vapor permeability through a material, such as a non-woven. The water vapor transmission rate indicates the breathability of the material. Permeability is a measure of the vapor moving through the material when there is equal static pressure on both sides of the material but the partial pressure is different (Frick's Law).

Referring now to FIGS. 8 and 9, an alternative embodiment is shown of an integral waistband 76 which can be formed on the front and/or back regions, 12 and/or 14 respectively, of a disposable absorbent article 10. The only difference in this embodiment from FIGS. 4 and 5 is the presence of at least one elastic member 78 secured to the non-laminate 64. In FIG. 8, three elastic members 78 are located on the non-laminate 64 and these three elastic members 78 are separated from the remaining elastic members 52 by a non-elastic region 80. The non-elastic region 80 is void or free of any elastic members. The non-laminate 64 is folded over the first end 56 of the outer layer 54 and the first end 46 of the inner layer 44 overlaps a portion of the outer layer 54. Again, the overlap portion is bonded to the outer layer 54 by an attachment mechanism 68, such as an adhesive. In FIG. 9, one can clearly see that the elastic members 78 are sandwiched between the inner and outer layers, 44 and 54 respectively, but are not present between the first surfaces 50 and 60. One will also notice that no two elastic members 52 or 78 overlap each other.

Referring now to FIGS. 10 and 11, still another embodiment is depicted of an integral waistband 82 which can be formed on the front and/or back regions, 12 and/or 14 respectively, of a disposable absorbent article 10. In this embodiment, one elastic member 84 is secured to the non-laminate 64 and this elastic member 84 is spaced away from the first end 46. Desirably, more than one elastic member 84 can be present. More desirably, from between about 3 to about 6 elastic members 84 are present. In FIG. 10, the elastic member 84 is secured to the first surface 50 of the inner layer 44 and is spaced apart from the first end 46 by a non-elastic region 86. The non-elastic region 86 is void or free of any elastic members. The non-laminate 64 is then folded over the first end 56 of the outer layer 54 and the first end 46 of the inner layer 44 overlaps a portion of the outer layer 54. Again, the overlap portion is bonded to the outer layer 54 by an attachment mechanism 68 to create a visible edge or waistband seam 72. Desirably, the attachment mechanism 68 is an adhesive. In FIG. 11, one can clearly see that the elastic member 84 is sandwiched between the fold 66 of the inner layer 44.

### Methods

A method of forming an integral waistband 70 on a disposable absorbent article 10 is described below. The integral waistband 70 can be formed only on the front region 12 of the disposable absorbent article 10, only on the back region 14 of the disposable absorbent article 10, or on both of the front and back regions, 12 and 14 respectively, so as to encircle the waist opening 38. The method first described will be for forming an integral waistband 70 only on the back region 14 of the disposable absorbent article 10. First, a front region 12 is formed having an inner layer 44 and an outer layer 54. At least one of the inner and outer layers, 44 and 54 respectively, can be formed from spunbond. Each of the inner and outer layers, 44 and 54 respectively, has a first end 46 and 56, a second end 48 and 58 and a first surface 50 and 60. At least two elastic members 52 are positioned adjacent to the first surface 50 of the outer layer 54 and the layers 44 and 54 are bonded together.

A back region 14 is formed having an inner layer 44 and an outer layer 54. At least one of the inner and outer layers, 44 and 54 respectively, can be formed from spunbond. Each of the inner and outer layers, 44 and 54, has a first end 46 and 56, a second end 48 and 58 and a first surface 50 and 60. The first end 46 of the inner layer 44 initially extends beyond the first end 56 of the outer layer 54. Desirably, the inner layer 44 is at least about 12 mm longer than the outer layer 54. More desirably, the inner layer 44 is from between about 25 mm to about 30 mm longer than the outer layer 54. At least two elastic members 52 are positioned adjacent to the first surface 60 of the outer layer 54. The first surface 60 of the outer layer 54 is then bonded to a portion of the first surface 50 of the inner layer 44 to form a laminate 62 and the remainder of the inner layer 44 is a non-laminate 64. The non-laminate 64 is folded over the first end 56 of the outer layer 54 and is secured to a portion of the outer layer 54, such as by an attachment mechanism 68, to form an integral waistband 70 on the back region 14. A stitch or design pattern can be added to the integral waistband 70, if desired, to make it more noticeable.

It should be recognized that the order of forming the front and back regions, 12 and 14 respectively, can be reversed or both regions 12 and 14 can be formed simultaneously, if desired.

An absorbent assembly 16 is then secured to the front and back regions, 12 and 14 respectively, approximate the second ends 48 and 58 of the inner and outer layers, 44 and 54 respectively. The front and back regions, 12 and 14 respectively, are joined together by a pair of seams 26 and 28 to form a unitary article 10 having a waist opening 38 and a pair of leg openings 40 and 42. The integral waistband 70 has a visible edge 72 located on the outer layer 54 of the back region 14 that is distally located away from the waist opening 38.

It should also be recognized that the non-laminate 64 can be folded over the first end 56 of the outer layer 54 after the absorbent assembly 16 is secured to the front and back regions, 12 and 14 respectively.

If one wishes to form the integral waistband 70 on the front region 12 of the disposable absorbent article 10 or around the entire circumference of the waist opening 38, this is also possible. It simply entails forming the front region 12 in a similar fashion as was explained above for forming the back region 14. It should also be noted that the integral waistband 70 or 70′ can stop short of the pair of seams 26 and 28 or the integral waistband 70 or 70′ can extend into one or both of the pair of seams 26 and 28.

It should further be recognized that one or more of the elastic members 52, 74 or 84 can be of a different color than the remaining elastic members. When colored elastic members 52, 74 or 84 are utilized, they should be visible through the inner or outer layers, 44 and 54 respectively. Desirably, the colored elastic members 74 and 84, located in the integral waistband 70, 70′ or 70″, will highlight the waistband and accentuate the presence of the integral waistband 70, 70′ or 70″ on the disposable absorbent article 10 or 10″.

In an alternative embodiment of the method, the inner layer 44 can be made from between about 10 to about 30 mm longer than the outer layer 54. The elastic members 52 are then positioned adjacent to the first surface 60 of the outer layer 54 and the outer layer 54 is bonded to the inner layer 44 to form a laminate 62. The inner layer 44 can be secured to the outer layer 54 by an adhesive, by ultrasonics, by heat, by pressure, by heat and pressure or by other means known to those skilled in the art. The portion of the inner layer 44 that extends beyond the first end 56 of the outer layer 54 is a non-laminate 64. Five elastic members 78 are secured to the non-laminate 64 and are spaced closer together than the remaining elastic members 52, located between the inner layer 44 and the outer layer 54. The top five elastic members 78 are of a different color than the remaining elastic members 52. For example, the top five elastic members 78 can be blue while the remaining elastic members 52 are white. The non-laminate 64 is then folded over the first end 56 of the outer layer 54 such that it overlaps a portion of the outer layer 54 and forms an integral waistband 76. The non-laminate 64 is bonded to the outer layer 54 and forms a visible edge or seam-like line 72 on the outer layer 54 away from the waist opening 38. A stitch or design pattern can be applied onto at least a portion of the folded over portion of the integral waistband 76 by using heat, pressure, heat and pressure or ultrasonics. The stitch or design pattern can highlight the visible edge or seam-like line 72. The pair of seams 26 and 28 used to create the waist opening 38 in the unitary disposable absorbent article 10 can be formed either before or after the folding step.

A further embodiment of the method involves making the inner layer 44 about 25 mm to about 30 mm longer than the outer layer 54. However, the left and right sides of the inner layer 44 are formed to be shorter than the remaining material. The elastic members 52 are again positioned adjacent to the first surface 60 of the outer layer 54 and the inner and outer layers, 44 and 54 respectively, are bonded together by an adhesive to form a laminate 62. The portion of the inner layer 44 that initially extends beyond the outer layer 54 is a non-laminate 64. Five elastic members 78 are secured on to the non-laminate 64 and are spaced closer together than the remaining elastic members 52 located between the inner and outer layers, 44 and 54 respectively. The top five elastic members 78 can be of a different color than the remaining elastic members 52. The non-laminate 64 is then folded over the first end 56 of the outer layer 54 and forms an integral waistband 76 having a visible edge 72. The visible edge 72 is spaced away from the waist opening 38. However, in this embodiment, the integral waistband 76 does not extend into the pair of seams 26 and 28 because the right and left sides of the non-laminate 64 were made shorter and no material was available to fold over the pair of seams 26 and 28.

As stated above, the integral waistband 76 can be secured to the outer layer 54 by an adhesive, ultrasonics, etc. In addition, a stitch or design pattern can be formed on the integral waistband 76 to highlight the visible edge or seam-like line 72.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

I claim:

1. An integral waistband for a disposable absorbent article having a waist opening, comprising:
a) an inner layer having a first end and a first surface;
b) an outer layer having a first end and a first surface; and
c) at least two elastic members being secured to said first surface of said outer layer, said inner and outer layers being bonded together such that said first end of said inner layer initially extends beyond said first end of said outer layer and the portion of said first surface of said inner layer bonded to said first surface of said outer layer forms a laminate and the remainder of said inner layer being a non-laminate, said non-laminate being folded over said first end of said outer layer and being bonded thereto to form an integral waistband having a visible edge located on said outer layer away from said waist opening.

2. The integral waistband of claim 1 wherein from between 2 to about 200 elastic members are present.

3. The integral waistband of claim 1 wherein at least four elastic members are present.

4. The integral waistband of claim 3 wherein said non-laminate overlaps at least two of said elastic members present in said laminate.

5. The integral waistband of claim 1 wherein each of said at least two elastic members is spaced apart from one another and said elastic member aligned closest to said first end of said outer layer is of a different color than at least one of said remaining elastic members.

6. The integral waistband of claim 1 wherein said inner and outer layers are formed from spunbond.

7. The integral waistband of claim 1 wherein said laminate is breathable.

8. The integral waistband of claim 1 wherein said integral waistband is breathable.

9. The integral waistband of claim 1 wherein at least one additional elastic member is secured to said non-laminate.

10. A disposable absorbent article having an integral waistband comprising:
a) a front region, a back region and an absorbent assembly secured to said front and back regions, said front and back regions being joined together by a pair of seams to form a unitary article having a waist opening, said front region including an inner layer having a first end, a second end and a first surface, an outer layer having a first end, a second end and a first surface, and a plurality of elastic members positioned adjacent to said first surface of said outer layer, said inner and outer layers being bonded together with said first end of said inner layer initially extending beyond said first end of said outer layer, the portion of said first surface of said inner layer bonded to said first surface of said outer layer forms a laminate and the remainder of said inner layer being a non-laminate, said non-laminate being folded over said first end of said outer layer and being bonded thereto to form an integral waistband aligned across said front region having a visible edge located on said outer layer away from said waist opening.

11. The disposable absorbent article of claim 10 wherein said integral waistband extends across at least a portion of said front region.

12. The disposable absorbent article of claim 11 wherein said integral waistband extends transversely across at least a portion of said front region.

13. The disposable absorbent article of claim 10 wherein said integral waistband extends completely across said front region between said pair of seams.

14. The disposable absorbent article of claim 10 wherein said integral waistband extends transversely across said front region and overlaps said pair of seams.

15. The disposable absorbent article of claim 10 wherein said plurality of elastic members includes at least three elastic members.

16. A disposable absorbent article having an integral waistband comprising:
a front region, a back region and an absorbent assembly bridging across said front and back region, said front and back regions being joined together by a pair of seams to form a unitary article having a waist opening and a pair of leg openings, said back region including an inner layer having a first end, a second end and a first surface, an outer layer having a first end, a second end and a first surface, and a plurality of elastic members positioned adjacent to said first surface of said outer layer, said inner and outer layers being bonded together with said first end of said inner layer initially extending beyond said first end of said outer layer, the portion of said first surface of said inner layer bonded to said first surface of said outer layer forms a laminate and the remainder of said inner layer being a non-laminate, said non-laminate being folded over said first end of said outer layer and being bonded thereto to form an integral waistband aligned across said back region having a visible edge located on said outer layer away from said waist opening.

17. A method of forming an integral waistband on a disposable absorbent article comprising the steps of:
a) forming a front region from an inner layer and an outer layer, each of said inner and outer layers having a first end, a second end and a first surface, said first end of said inner layer initially extending beyond said first end of said outer layer, and positioning a plurality of elastic members adjacent to said first surface of said outer layer and bonding said layers together;
b) forming a back region from an inner layer and an outer layer, each of said inner and outer layers having a first end, a second end and a first surface, said first end of said inner layer initially extending beyond said first end of said outer layer, and positioning a plurality of elastic members adjacent to said first surface of said outer layer and bonding said first surface of said outer layer to a portion of said first surface of said inner layer to form a laminate and the remainder of said inner layer being a non-laminate;
c) securing an absorbent assembly to said front and back regions approximate said second ends of said inner and outer layers;
d) joining said front and back regions together by a pair of seams to form a unitary article having a waist opening and a pair of leg openings; and
e) folding and bonding said non-laminate over a portion of said outer layer of said back region to form an integral waistband having a visible edge located on said outer layer of said back region away from said waist opening.

18. The method of claim 17 wherein said integral waistband contains colored elastic members.

19. The method of claim 17 further includes making at least one of said inner and outer layers out of spunbond.

20. The method of claim 17 further includes forming said inner layer at least about 12 millimeters longer than said outer layer.
